(19) United States
(12) Patent Application Publication
(10) Pub. No.: US 2009/0239891 A1
(43) Pub. Date: Sep. 24, 2009

(54) Sustained Release Dosage Forms of Analgesic Medications

(76) Inventors: Atul J. Shukla, Cordova, TN (US); James R. Johnson, Germantown, TN (US); Yichun Sun, Gaithersburg, MD (US); Yingxu Peng, Pennington, NJ (US); Shipeng Yu, Memphis, TN (US); Wen Qu, Memphis, TN (US); Timothy D. Mandrell, Memphis, TN (US)

Correspondence Address: Howard Eisenberg, Esq., 1220 Limberlost Lane, Gladwyne, PA, 19035, US

(21) Appl. No.: 12/087,692

(22) PCT Filed: Feb. 28, 2007
(86) PCT No.: PCT/US07/05379

§ 371 Date: Dec. 18, 2008

Related U.S. Application Data
(60) Provisional application No. 60/777,913, filed on Mar. 1, 2006.

(51) Int. Cl. A61K 31/4355; A61K 31/445

(52) U.S. Cl. 514/279; 514/329

(57) ABSTRACT

Parenteral extravascular administration of a composition containing an analgesic medication of low water solubility that is dissolved, suspended, or emulsified in a solvent system results in the deposition of the analgesic medication at the site of administration and provides a controlled release of the analgesic medication from the site and a prolonged analgesia that may persist for several days following administration.

### FIELD OF THE INVENTION

This invention pertains to the field of the provision of analgesic medications to prevent or reduce acute or chronic pain and particularly to sustained release dosage forms for analgesic medications.

### BACKGROUND OF THE INVENTION

Chronic pain occurs with many conditions affecting humans and veterinary patients. It is associated with a great variety of conditions, including cancer, arthritis, low-back pain, repetitive stress, neurologic injury or disease, trauma, and following surgery. Chronic pain may in extreme circumstances become debilitating for an individual suffering from the pain.

Laboratory animals are often subjected to various painful surgical procedures such as laparatomy, thoracotomy, or orthopedic procedures as well as non-surgical procedures such as the induction of arthritis. It is a paramount ethical obligation of all research personnel involved with the care and use of laboratory animals to reduce or preferably eliminate pain and distress by using analgesics, provided these analgesics do not interfere with the research objectives.

The most widely used laboratory animals for various types of research involving painful procedures are rodents, such as rats, mice, and guinea pigs. A survey of literature indicates that the opioid buprenorphine is the most widely used narcotic analgesic for rodents because of its excellent analgesic activity and duration of action. Moreover, unlike morphine, respiratory depression is not usually a problem with this opioid. However, in order to provide adequate analgesia, repeated parenteral administration is required, typically by subcutaneous injection, a process that is extremely stressful to the animals.

In order to reduce the frequency of handling and in turn, improve the well being of the animals under treatment, a sustained release formulation of an analgesic, such as buprenorphine, butorphanol, or fentanyl, or a local anesthetic such as bupivacaine, that is capable of maintaining analgesia in laboratory animals for 3 to 5 days following a single administration of a drug loaded formulation would be highly desirable. Such a long-term analgesic formulation, which would reduce stress in both laboratory animals undergoing painful procedures and in personnel who administer analgesic formulations to these animals, does not currently exist.

Buprenorphine is an opioid medication with partial agonist and antagonist actions. It has been used as an analgesic in humans and in non-human animals, and is used extensively for analgesia in laboratory animals. In humans, it has also been used to treat opioid dependence. Buprenorphine is marketed as a water-soluble hydrochloride salt. No long-duration form of an injectable buprenorphine hydrochloride is available, although a solid implant of buprenorphine hydrochloride has been reported. Pontani and Misra, Pharmacology, Biochemistry and Behavior, 18(3):471-474 (1983).

In addition to laboratory animals, long-term analgesia following an administration of an analgesic medication is needed in human and non-human animals for management of acute and chronic pain. In human and veterinary patients, such as those suffering from long-term chronic pain due to surgeries or illnesses such as arthritis or cancer, a sustained release formulation requiring administration only every three to five days or longer would increase compliance and would reduce the burden on the patient and on caregivers.

In humans, a sustained release formulation of an opioid drug such as buprenorphine would also be of considerable benefit in the treatment of addiction to opioid drugs such as morphine or heroin.

A significant need exists, therefore, for a sustained release formulation of an analgesic medication, such as an opioid analgesic or a local anesthetic, that can be administered parenterally and which maintains its analgesic effect upon administration for a period of 24 hours or longer, or preferably for a period of three to five days or even longer.

### BRIEF DESCRIPTION OF THE DRAWINGS

FIG. 1 (Prior Art) is a semi-log plot of the time course of the plasma concentration after intravenous administration of 2.4 mg/kg (base equivalents) of buprenorphine hydrochloride intravenously into the tail vein of mice. Data points represent the mean±standard deviation of four mice at each time point. The solid line represents the predicted plasma concentration based on the pharmacokinetic parameter estimates from the three-compartment model.

FIG. 2 (Prior Art) is a line graph of the percentage of maximum possible analgesic effect (% MPE) obtained in mice after an intravenous bolus injection of 2.4 mg/kg (base equivalents) of buprenorphine hydrochloride solution.

FIG. 3 is a graph of the analgesic effect (% MPE) obtained in mice after a subcutaneous injection of a suspension of buprenorphine free base in TEC. The dose of buprenorphine was approximately 2 mg/mouse.

FIG. 4 is a graph of the analgesic effect (% MPE) obtained in mice after a subcutaneous injection of a suspension of buprenorphine free base in ATEC. The dose of buprenorphine was approximately 2 mg/mouse.

FIG. 5 is a graph of the analgesic effect (% MPE) obtained in mice after a subcutaneous injection of a suspension of buprenorphine free base in Na CMC. The dose of buprenorphine was approximately 4 mg/mouse.

FIG. 6 is a graph of the analgesic effect (% MPE) obtained in mice after a subcutaneous injection of a buprenorphine suspension of free base in PEG 400. The dose of buprenorphine was approximately 4 mg/mouse.

FIG. 7 is a graph of the analgesic effect (% MPE) obtained in mice after a subcutaneous injection of a suspension of buprenorphine free base in TEC. The dose of buprenorphine was approximately 4 mg/mouse.

FIG. 8 is a graph of the analgesic effect (% MPE) obtained in mice after a subcutaneous injection of a suspension of buprenorphine free base in ATEC. The dose of buprenorphine was approximately 4 mg/mouse.

FIG. 9 is a graph of the analgesic effect (% MPE) obtained in mice after a subcutaneous injection of a suspension of buprenorphine free base in TBC. The dose of buprenorphine was approximately 4 mg/mouse.

FIG. 10 is a graph of the analgesic effect (% MPE) obtained in mice after a subcutaneous injection of a suspension of buprenorphine free base in ATBC. The dose of buprenorphine was approximately 4 mg/mouse.

FIG. 11 is a graph of the analgesic effect (% MPE) obtained in mice after a subcutaneous injection of a suspension of buprenorphine free base in sesame oil. The dose of buprenorphine was approximately 4 mg/mouse.

FIG. 12 (Prior Art) is a graph of the plasma concentration of fentanyl after an intravenous injection of a fentanyl solution.

FIG. 13 is a graph of the plasma concentration of fentanyl in dogs following a subcutaneous injection of a fentanyl base/tetraglycol/water solution at a dose of 4 mg per dog.

FIG. 14 is a graph of the plasma concentration of fentanyl in dogs following a subcutaneous injection of a fentanyl base/PEG/water solution at a dose of 4 mg per dog.

FIG. 15 is a graph of the plasma concentration of fentanyl in dogs following a subcutaneous injection of a fentanyl base/ATBC solution at a dose of 4 mg per dog.

FIG. 16 is a graph of the plasma concentration of fentanyl in dogs following a subcutaneous injection of a fentanyl base/tetraglycol/water solution at a dose of 8 mg per dog.

FIG. 17 is a graph of the plasma concentration of fentanyl in dogs following a subcutaneous injection of a fentanyl base/ATBC solution at a dose of 8 mg per dog.

### DESCRIPTION OF THE INVENTION

It has been discovered that parenteral extravascular administration of a composition containing an analgesic with low water solubility at physiological pH that is dissolved, suspended, or emulsified in a solvent system results in a depot of the analgesic medication at the site of administration and provides for a controlled or sustained release of the analgesic medication from the site and a resultant prolonged analgesia that may persist for several days following a single administration. The compositions and methods of the invention are useful in providing sustained release of a medication to provide long term analgesia in humans and other animals and may also be useful in the treatment of addiction to analgesic medications such as opioids.

As used herein, the term “parenteral extravascular administration” means administration of a pharmaceutical composition by injection or implantation within the body, which administration is not within the venous or articular circulation. That is, the administration is within the body in a location that is outside of the gastrointestinal tract distal to the mouth and proximal to the anus and is outside of the cardiovascular system. For example, the administration according to the invention may be in or beneath the skin or an externally accessible mucous membrane such as the oral, vaginal, or anal mucosa, within a striated muscle, or into a wound or cavity created by trauma such as surgical or non-surgical trauma.

As used herein, the term “low water solubility” means that a medication is sparingly or less soluble in water. That is, the term “low water solubility” when referring to a medication means that the medication is “sparingly soluble” (30 to 100 parts of water needed to dissolve 1 part of the medication), is “slightly soluble” (100 to 1000 parts of water needed to dissolve 1 part of the medication), is “very slightly soluble” (1000 to 10,000 parts of water needed to dissolve 1 part of the medication), or is “practically insoluble or insoluble” (more than 10,000 parts of water needed to dissolve 1 part of the medication) in water.

As used herein, the term “solvent system” refers to one or more solvents (i.e. mixture of solvents) with or without co-solvents in which a medication is dissolved, emulsified, or suspended within a composition. The term “solvent system” may also refer to a vehicle (containing one or more materials) in which the medication is dissolved, suspended, or emulsified. The multiplicity of solvents in a solvent system may or may not be miscible with one another and may include solvents of varying polarities or hydrophilicities and hydrophobicities.

In one embodiment, the invention is a composition for long-term analgesia in a human or non-human animal. According to this embodiment of the invention, the composition contains an analgesic with low water solubility at physiological pH that is dissolved, suspended, or emulsified in a solvent system. In a preferred embodiment, the composition is situated in situ within the body of an animal, preferably in a parenteral, extravascular site. In a preferred embodiment, the analgesic medication is an opioid analgesic such as buprenorphine or fentanyl.

Besides the solvent system, the above mentioned composition may or may not contain other additives such as natural, semi-synthetic or synthetic additives such as polymers, waxes, gums, resins, surfactants, and chelating agents, that modify and/or modulate either deposition and/or partitioning of the analgesic at the site of administration.

In another embodiment, the invention is a composition in the form of a solution of the analgesic medication, such as a local anesthetic. The solution of an analgesic with low water solubility at physiological pH can be prepared by dissolving the analgesic in a hydrophilic solvent system or a hydrophobic solvent system with or without heating.

If a hydrophilic solvent system is used to prepare the solution, then upon administration, the analgesic will be deposited at the site of administration when the analgesic solution comes in contact with aqueous body fluid. The deposited analgesic is then slowly dissolved in the aqueous medium at the site of administration to provide sustained analgesia that persists for several days following administration.

If a hydrophobic solvent system is used to prepare the solution, the controlled release of the analgesic from the hydrophobic solvent system is conceived to be predominantly due to partitioning of the analgesic between the hydrophobic solvent system and the body fluid. In addition to this partitioning, it is conceived that the release of the analgesic will also occur due to dissolution of the solid particles of the analgesic (that are deposited at the site of administration) in the aqueous body fluid at the administration site. The partitioned and/or dissolved analgesic is then absorbed from the aqueous body fluid at the site of administration to provide sustained analgesia that may persist for several days following administration.

The solution of the analgesic medication of low water solubility may be produced by dissolving the medication in a hydrophilic solvent system that may be composed of a single or a blend of two or more solvents. The hydrophilic solvent system may be:

1) a water miscible solvent or
2) a blend of two or more water-miscible solvents, or
3) a blend of a water miscible solvent and water, or
4) a blend of two or more water-miscible solvents.

Alternatively, the solvent system may be a blend of two or more polar and/or non-polar solvents, i.e. solvents of varying hydrophilicities and hydrophobicities, to obtain an optimum solvent system which can control/optimize the rate of deposition of the analgesic with low water solubility at physiological pH at the administration site. More than two solvents with varying polarities or hydrophilicity and hydrophobicity may also be mixed, such that overall, the resulting solvent system is of the desired hydrophilicity or polarity which can control/optimize the rate of release of the analgesic with low water solubility at physiological pH from the solvent system at the administration site and provide sustained analgesia that may persist for several days following administration.

The aforementioned solvent system used to dissolve the analgesic with low water solubility at physiological pH may also be hydrophobic and may be:

1) a non-polar or a hydrophobic solvent, or
2) a blend of two or more hydrophobic solvents.

Alternatively, the solvent system may also be a blend of two or more non-polar and polar solvents, i.e. a blend of solvents of varying hydrophobicities and hydrophilicities to obtain an optimum hydrophobic solvent system which can control/optimize the rate of release of the analgesic with low water solubility at physiological pH from the solvent system at the administration site and provide sustained analgesia that may persist for several days following administration.

Additionally, the composition may contain additives such as polymers, waxes, gums, resins, surfactants, chelating agents, or others that modify and/or modulate either deposition and/or partitioning of the analgesic at the site of administration, and these additives may be added to the solution prepared with either hydrophilic or hydrophobic solvent system in order to modulate the release kinetics of the analgesic from the administered solution, thus providing sustained analgesia that may persist for several days following administration.

In another embodiment, the invention is a composition in the form of a suspension of the analgesic medication, such as a local anesthetic. The suspension of an analgesic with low water solubility at physiological pH may be prepared by mixing fine particles of the analgesic of low water solubility at physiological pH with a hydrophilic solvent system or a hydrophobic solvent system.

Suspension of the analgesic may be prepared in vitro by precipitating the analgesic by mixing a solution of the analgesic in an appropriate solvent system with a second solvent system which is miscible with the solvent system used to dissolve the analgesic but in which the analgesic has low solubility, as defined above. Such a suspension may be prepared from a solution immediately prior to administration or may be prepared in advance of administration and may be packaged in individual or multiple use containers from which an appropriate dose may be removed at the time of administration.

The abovementioned solution of the analgesic used for preparation of the suspension may be prepared by using a water-miscible solvent or solvents. The second solvent for the analgesic may be water or a blend of water and other solvents which are miscible with the solvent or solvents used to dissolve the analgesic. Suspensions of the analgesic in both hydrophilic and hydrophobic solvent systems can be prepared by the abovementioned method.

If a hydrophilic solvent system is used to prepare the suspension, the hydrophilic solvent system will be removed from the administration site by the aqueous medium at the site of administration when the analgesic suspension comes in contact with the aqueous body fluid. The deposited analgesic is then dissolved slowly in the aqueous medium at the site of administration to provide sustained analgesia that may persist for several days following administration.

If a hydrophobic solvent system is used to prepare a suspension, the release of the analgesic from the suspension into the aqueous body fluid at the site of administration could occur by partitioning of the analgesic that is dissolved in the hydrophobic solvent, dissolution of the solid analgesic particles or both. The released analgesic is then absorbed from the aqueous body fluid at the site of administration to provide sustained analgesia that may persist for several days following administration.

The suspension of the analgesic may be produced by dispersing the analgesic in a hydrophilic solvent system that may be composed of a single or a blend of two or more solvents. The hydrophilic solvent system may be:

1) water, or
2) a water miscible solvent, or
3) a blend of two or more water-miscible solvents, or
4) a blend of a water miscible solvent and water, or
5) a blend of two or more water-miscible solvents and water.

Alternatively, the solvent system may be a blend of two or more polar and/or non-polar solvents, i.e. solvents of varying hydrophilicities and hydrophobicities to obtain an optimum solvent system which can control/optimize the rate of deposition of the analgesic at the administration site. More than two solvents with varying polarities or hydrophilicity and hydrophobicity can also be mixed, such that overall, the resulting solvent system is of the desired hydrophilicity, which will cause the analgesic to be deposited at the site of administration and/or partitioned into aqueous body fluid at the site of administration, at a controlled rate. This deposited analgesic at the site of administration will then dissolve slowly because of the slow dissolution rate of the analgesic. Partitioning of the analgesic and/or slow absorption of the analgesic from the administration site will also provide prolonged analgesia that may persist for several days following administration.

The solvent system used to disperse the analgesic with low water solubility at physiological pH to prepare a suspension could also be hydrophobic and may include the following:

1) a non-polar or a hydrophobic solvent, or
2) a blend of two or more hydrophobic solvents.

Alternatively, the hydrophobic solvent system may be a blend of two or more non-polar and polar solvents, i.e. solvents of varying hydrophobicities and hydrophilicities to obtain an optimum hydrophobic solvent system which can control/optimize the rate of release of the analgesic with low water solubility at physiological pH from the suspension (though dissolution and/or partitioning) at the administration site and provide sustained analgesia that may persist for several days following administration.

Additionally, natural, semi-synthetic or synthetic additives such as polymers, waxes, gums, resins, surfactants, chelating agents, or others that modify and/or modulate either deposition and/or partitioning of the analgesic at the site of administration, can be added to the suspension prepared with either hydrophilic or hydrophobic solvent system to modulate the release kinetics of the analgesic from the administered suspension, thus providing sustained analgesia that may persist for several days following administration.

In another embodiment, the invention is a composition in the form of an emulsion containing the analgesic medication. The emulsion of the medication can be prepared by emulsifying a solution of the analgesic or local anesthetic with low water solubility at physiological pH in a hydrophobic solvent system (dispersed phase) with a solvent system (dispersion medium), which is immiscible with the hydrophobic solvent system, and an appropriate additive such as a surfactant. The hydrophobic solvent system or the dispersed phase may include the following:

1) a non-polar or a hydrophobic solvent, or
2) a blend of two or more hydrophobic solvents, or
3) a blend of a hydrophobic solvent and a hydrophilic solvent, whereby the resulting solvent blend is hydrophobic.

The immiscible solvent system or the dispersion medium may include one of the following:

1) water, or
2) a water miscible solvent, or
3) a blend of two or more water-miscible solvents, or
4) a blend of a water miscible solvent and water, or
5) a blend of two or more water-miscible solvents and water.

In addition, natural, semi-synthetic or synthetic additives such as thickening agents, gums, polymers, waxes, resins, clays, surface or interfacial acting agents, or others can be added into either the dispersed phase or the dispersion medium or both, to modulate the release characteristics of the analgesic. The emulsion forms a depot at the site of administration and slowly releases the analgesic into the body fluid to provide sustained analgesia that may persist for several days following administration.

In another embodiment, the invention is a method for producing long-term analgesia in animals. According to this embodiment of the invention, a composition containing an analgesic with low water solubility at physiological pH that is dissolved, suspended, or emulsified in a solvent system is administered parenterally and extravascularly into the body of an animal in need thereof. In a preferred embodiment, the analgesic medication is an opioid analgesic such as buprenorphine or fentanyl.

In another embodiment, the invention is a method for obtaining an in situ depot of a medication, such as an analgesic medication of low water solubility at physiological pH, such as an opioid like buprenorphine or fentanyl, by administrating a solution of the aforementioned medications in a water-miscible solvent system, in which the medications have high solubility, into human or animals. According to this embodiment of the invention, the depot is obtained at the administration site because of the precipitation of the medication from the solution. Upon parenteral and extravascular administration, the medication in the solution forms a depot at the application site and slowly releases/dissolves into the body fluid.

In another embodiment, the invention is a method for obtaining an in situ depot of a medication, such as an analgesic medication of low water solubility at physiological pH, such as an opioid like buprenorphine or fentanyl, by administrating a suspension of the aforementioned medications in a water-miscible solvent system, in which the medications have low solubility, into human or animals. According to this embodiment of the invention, the depot is obtained at the administration site because of deposition of the medication from the suspension. Upon parenteral and extravascular administration, the medication in the suspension forms a depot at the application site and slowly releases/dissolves into the body fluid.

In another embodiment, the invention is a method for obtaining an in situ depot of a medication, such as an analgesic medication of low water solubility at physiological pH, such as an opioid like buprenorphine or fentanyl, by administrating a suspension of the aforementioned medications in a water-immiscible solvent system, in which the medications have low solubility, into human or animals. According to this embodiment of the invention, the depot is obtained at the administration site because of deposition of the medication from the suspension. Upon parenteral and extravascular administration, the medication in the suspension forms a depot at the application site and slowly releases by partitioning and/or dissolving into the body fluid.

In another embodiment, the invention is a method for obtaining a suspension of a medication, such as an analgesic medication of low water solubility at physiological pH, such as an opioid like buprenorphine or fentanyl, in an aqueous medium such as saline solution containing sodium CMC and Tween 80. According to this embodiment of the invention, the suspension is obtained by mixing fine particles of a medication of low water solubility with an aqueous medium (solvent system) to obtain a suspension of the medication. Upon parenteral and extravascular administration, the medication in the suspension forms a depot at the application site and slowly releases into the body fluid.

In another embodiment, the invention is a method for obtaining a suspension of a medication of low water solubility, such as an analgesic medication, such as an opioid like buprenorphine or fentanyl, in a water-immiscible medium such as vegetable oil containing suitable additives. According to this embodiment of the invention, the suspension is obtained by mixing fine particles of a medication of low water solubility with a water-immiscible solvent system such as an oil like a vegetable oil. The medication in the suspension forms a depot at the application site and slowly release into the body fluid. The release of the medication from such a suspension is predominantly controlled by partitioning between the solvent and the body fluid. However, dissolution of the medication particles may become a predominant release mechanism after the absorption of the water-immiscible solvent system such as vegetable oil.

In another embodiment, the invention is a method for obtaining a solution of a medication of low water solubility at physiological pH, such as an analgesic medication, such as an opioid like buprenorphine or fentanyl, in a water-immiscible medium such as vegetable oil containing suitable additives. According to this embodiment of the invention, the solution is obtained by mixing a medication with low water solubility with a water-immiscible solvent system with or without heating. The medication in oil solution forms a depot at the application site and slowly release into the body fluid. The release of the medication from such oily solution is predominantly controlled by partitioning between oil and the body fluid. Additionally, a solvent or solvents or additives can be added into the solution to modulate the partition kinetics of the medication in order to modulate the release characteristics of the medication.

In another embodiment, the invention is a method for obtaining an emulsion of an medication with low water solubility at physiological pH, such as an opioid like buprenorphine or fentanyl. According to this embodiment of the invention, the emulsion is obtained by emulsifying the solution of the medication with low water solubility at physiological pH in a hydrophobic solvent system such as an oil, with a solvent system that is immiscible with the hydrophobic solvent system, and an appropriate additive such as an emulsifier such as a surfactant. Additionally, a solvent or mixtures of solvents with or without additives can be added into the dispersed phase in the emulsion or the dispersion medium to modulate the release characteristics of the medication. The emulsion forms a depot at the site of administration and slowly releases the analgesic into the body fluid to provide sustained analgesia that may persist for several days following administration.

In another embodiment, the invention is a method for the treatment of opiate addiction in humans. According to this embodiment of the invention, a sustained release injectable dosage form of an opioid agonist medication, such as buprenorphine, is administered parenterally and extravascularly for the treatment of opiate addiction in human beings. The sustained release nature of the injected dosage forms is associated with improved patient compliance.

The invention is disclosed herein primarily with reference to opioid agonists such as buprenorphine and fentanyl. However, one skilled in the art will recognize that the invention is applicable to other analgesic medications of low water solubility at physiological pH including opioid and non-opioid analgesic medications, and local anesthetics. Analgesics with low water solubility at physiological pH may include free acids, free bases, and salt forms, preferably other than a hydrochloride or a sulfate, of a drug with low aqueous solubility, particularly at physiological pH. Such opioid analgesics fall into three classes. Phenanthrene opioid analgesics include morphine, hydromorphone, oxymorphone, levorphanol, codeine, hydrocodone, butorphanol, nalbuphine, pentazocine, and dezocine. Pehylpiperidine opioid analgesics include meperidine, fentanyl, sufentanil, and alfentanil. Phenylheptanone opioid analgesics include methadone, propoxyphene, and levomethadyl. An example of a non-opioid analgesic is tramadol. Therefore, in this specification, the disclosures concerning buprenorphine may be applied to these other opioid and non-opioid analgesic medications. Also included within the scope of the invention and within the definition of the term “analgesic” for the purpose of this application are local anesthetics, as such anesthetics may also be used in depot formulations for the purpose of localized pain management.

The solvent system of the composition of the invention may be composed of any solvent in which an analgesic medication may be dissolved, suspended, or emulsified, either alone or with additional solvents and/or co-solvents that comprise the solvent system. Illustrative examples of solvents include water, oil such as a vegetable oil like sesame oil, corn oil, or soybean oil, citric acid esters such as triethyl citrate (TEC), acetyl triethyl citrate (ATEC), tributyl citrate (TBC), acetyl tributyl citrate (ATBC), n-methylpyrrolidone 2-pyrrolidone (NMP), tetraglycol, propylene glycol, and polyethylene glycol (PEG).

The composition of the invention may be in the form of a suspension which is prepared by mixing an analgesic medication, such as buprenorphine or fentanyl, with an appropriate solvent. For example, a buprenorphine suspension may be obtained by mixing buprenorphine base or a salt of buprenorphine with low aqueous solubility, with water, such as reverse osmosis (RO) water, which may contain a surfactant, such as polysorbate 20. The suspensions may also contain a suspending agent, such as sodium carboxymethylcellulose.

An alternative method for obtaining suspension solid particles of a medication of low-water solubility, such as an analgesic medication such as buprenorphine, at the site of administration is to precipitate the medication from a solution of the medication in a solvent system in which the medication has high solubility by exposing the solution to an aqueous environment. Such exposure may be in vitro, by mixing the solution with water and/or an aqueous solvent. As an example, approximately 1.8 mg of buprenorphine base was dissolved in 40 mg of n-methylpyrrolidone. The buprenorphine in the resulting solution precipitated and formed a suspension upon addition of 80 microliters of water, such as RO water, into the solution. Alternatively, such exposure may be in vivo which results in in situ precipitation of the medication at the application sites.

Distinct advantages of preparing a suspension or precipitating a drug from a solution or emulsion, as illustrated above, either in vitro or in vivo, include:

1) Accurate dosing of the drug if it is injected as a solution or emulsion and the drug is allowed to precipitate at the injection site or partition out of the emulsion in vivo;
2) Ease of preparation of a long-acting suspension of a drug, such as an analgesic medication such as buprenorphine or fentanyl, if the drug is precipitated to form a suspension in vitro;
3) Formation of small particle size of the drug, such as an analgesic medication such as buprenorphine or fentanyl, by first dissolving the drug in a solvent in which the drug has high solubility and then precipitating the drug in vitro by mixing the solution with a solvent in which the drug has poor solubility.

Solution of medications, such as analgesic medications like buprenorphine or fentanyl, that are prepared with a water-immiscible solvent system, such as comprising tributyl citrate or oil, that remain at an injection site for a prolonged period of time, can sustain the release of the medication into the body. If desired, the non-aqueous solution may be further dispersed into a solvent that is immiscible with the non-aqueous solvent, such as water, to form an emulsion as a final dosage form. For example, 1 ml of buprenorphine in tributyl citrate solution (approximately 50 mg/ml) may be dispersed into 2 ml of RO water containing 0.5% of a surfactant such as TWEEN 80. The resulting emulsion may be injected, such as subcutaneously. Drug release from the administered solutions or emulsions is conceived to be due primarily to partitioning of the medication from the non-aqueous solution or from the emulsion to the aqueous environment within the body at the site of administration.

In a preferred embodiment, the sole constituents of the suspension, solution, or emulsion of the invention are the analgesic medication, such as buprenorphine or fentanyl, and a solvent system, which may be a combination of a multiplicity of solvents and may include co-solvents. That is, preferably, 100% of the constituents of the formulation other than the analgesic medication is composed of the solvent system. In a less preferred embodiment, less than 100% of the constituents of the formulation other than the medication is composed of the solvent system.

Other ingredients may optionally be included in the composition of the invention. These optional ingredients may include colorizing agents, polymeric or non-polymeric thickeners/suspending agents/gelling agents, buffers, preservatives, additional aqueous or non-aqueous solvents, stabilizers, antioxidants, surfactants, emulsifiers, and chelating agents. Examples of thickener/suspending/gelling agents that may be included in the formulation include natural, synthetic, or semi-synthetic polymers such as carboxymethylcellulose (CMC), polyvinlypyrrolidone (PVP), polyvinyl alcohol (PVA), and polyethylene glycols (PEG), magnesium aluminum silicate (Veegum®, R. T. Vanderbilt Co., Inc., Norwalk, Conn.), Carbopol® 934 (Noveon Inc., Cleveland, Ohio), gums such as acacia, tragacanth, guar gum, locust bean gum, bentonite, aluminum stearate and colloidal silica (SiO₂), waxes (natural, synthetic or semisynthetic), biodegradable polymers, or a combination of these.

The composition of the invention may be utilized for obtaining long-term analgesia in human and non-human animals, such as non-human mammals, birds, and reptiles. The animals of the invention may be domestic animals, such as dogs, cats, horses, cattle, or captive birds or may be non-domestic animals such as wild or captive animals. In humans and veterinary patients, the compositions and methods of the invention may be utilized for pain management, such as for pain following surgery or pain due to chronic illnesses such as cancer.

In a preferred embodiment, the animals of the invention are laboratory animals, such as birds, dogs, cats, rabbits, non-human primates, and rodents such as mice, rats, guinea pigs, gerbils, and hamsters. According to this embodiment of the invention, a sustained release injectable dosage form of an analgesic medication, such as buprenorphine or fentanyl, is administered parenterally to an animal for the purpose of pain management. The pain may be due to a treatment that is inflicted upon the animal by a human, such as during a scientific study or experiment. Alternatively, the pain may be due to a natural cause, such as an injury or an illness such as arthritis.

According to this embodiment of the invention, a solution, suspension, or emulsion composition of the invention as described above is administered parenterally and extravascularly to an animal in need thereof. This embodiment of the invention obviates the need to administer an analgesic medication at frequent intervals, such as one or more times daily. According to the administration embodiment of the invention, administration of the analgesic medication composition of the invention provides analgesia that lasts for several days, typically 3 to 5 days or longer depending on the need.

Administration of the composition of the invention is parenteral and extravascular, preferably by subcutaneous injection. Other routes and means of administration may also be utilized provided that such route permits a bolus of the administered composition to remain in situ for a time sufficient for the analgesic medication of the composition to precipitate from the administered suspension, solution, or emulsion. Such routes and means include injection and implantation, such as within a body cavity or muscle, or under or within the skin or a mucosal surface such as within the mouth.

To further illustrate the invention, the following examples are provided. It is to be understood that these examples are provided for illustrative purposes and are not to be construed as limiting the scope of the invention.

### EXAMPLE 1

### Prior Art—Plasma Concentration Following Intravenous Administration

Forty eight female CD-1 mice weighing 25-30 grams randomly assigned into 12 groups of four mice. Each mouse was held in a restraint system and administered a 2.4 mg/kg dose (buprenorphine base equivalents) of buprenorphine hydrochloride intravenously into the tail vein over 5 seconds. Blood samples (˜1 ml) were collected in heparinized 1.5 ml centrifuge tubes by cardiac puncture under heavy, isoflurane general anesthesia at 5, 15, and 30 minutes, and 1, 2, 3, 5, 7, 9, 12, 18, and 24 hours after drug administration. For each time point, four mice were sacrificed, and plasma (0.5 ml) was separated by centrifugation at 1100×g for 10 minutes, and stored at −70° C. until analysis. The concentration of buprenorphine in the mouse plasma samples was determined according by liquid chromatographic-tandem mass spectrometry (LC/MS).

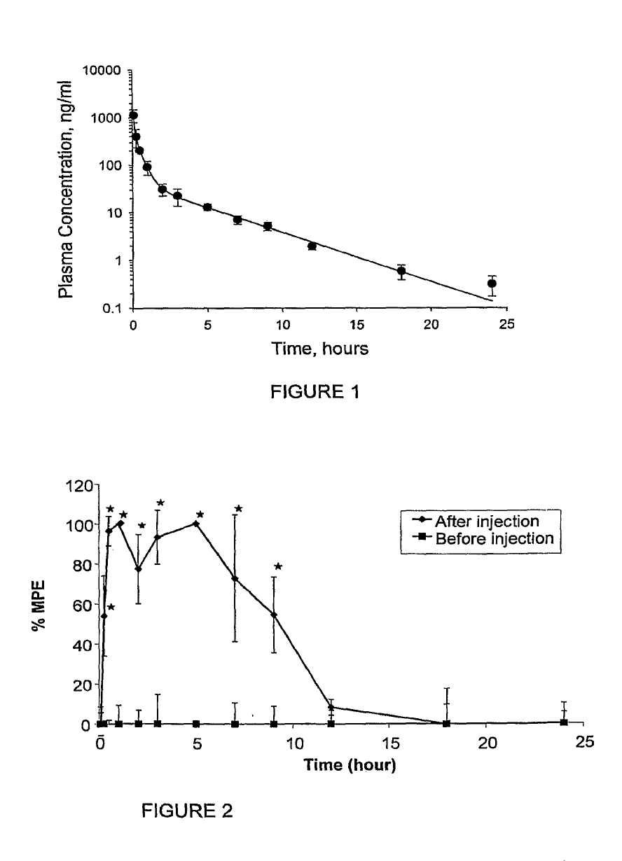

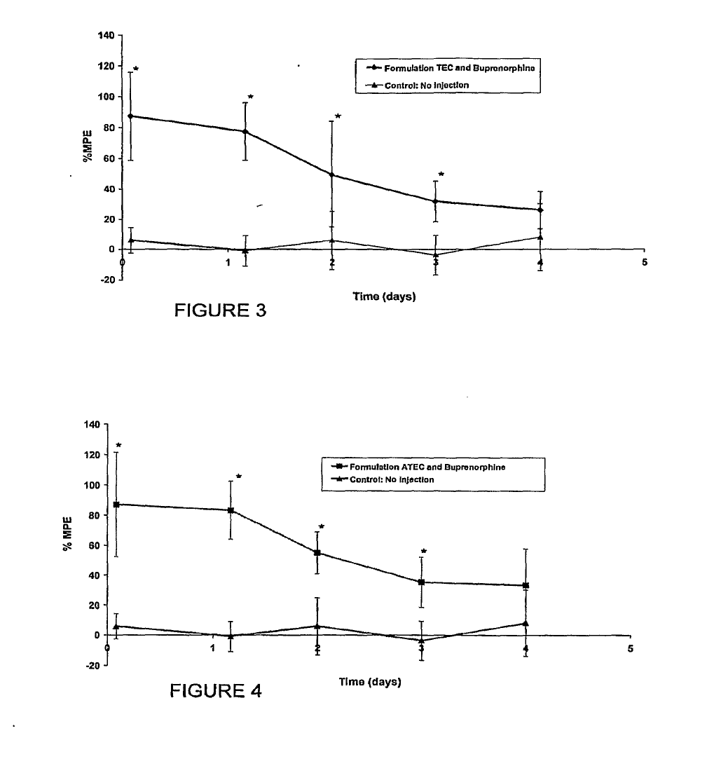

The plasma concentration-time profile of observed and model predicted buprenorphine concentrations following an intravenous administration of 2.4 mg/kg is shown in FIG. 1. As shown, plasma concentration of buprenorphine following intravenous administration rapidly decreased in the first minutes following administration and, within 5 or 6 hours, the plasma concentration had been reduced to approximately 1% of the initial concentration.

### EXAMPLE 2

### Prior Art—Duration of Analgesia Following Intravenous Administration

Forty eight female CD-1 mice weighing 25-30 grams randomly assigned into 12 groups of four mice. Each mouse was held in a restraint system and administered a 2.4 mg/kg dose (buprenorphine base equivalents) of buprenorphine hydrochloride intravenously into the tail vein over 5 seconds. The analgesic effect of the administered buprenorphine was measured by the tail flick method at predetermined time points.

The analgesic effect at each time point after injection was reported as the Percentage of Maximum Possible Effect (% MPE). This method compares the latency at each time point to the animal's own average baseline latency. The % MPE was then calculated using the following formula:

$$\% M P E = \frac{Latency - Baseline Latency}{Cutoff Time - Baseline Latency} \times 100 \%$$

The results are shown graphically in FIG. 2. As shown, the onset of analgesic effect was 15 minutes after the intravenous injection, with 53.7% MPE (P<0.05 compared to % MPE before injection). The analgesic effect increased from 53.7% MPE to 100% MPE at 60 minutes and remained at elevated levels ranging from 70%˜100% MPE (P<0.05 compared to corresponding % MPE before injection) until 7 hours after injection. The analgesic effect dropped to 54% MPE (P<0.05 compared to corresponding % MPE before injection) at 9 hours after injection, and then dropped to the 0% MPE (P>0.05 compared to corresponding % MPE before injection) at 12 hours. No analgesic effect for the drug was observed between 12 and 24 hours after injection.

Table 1 shows the plasma concentration of buprenorphine as determined in Example 1 and the corresponding analgesic effect achieved after intravenous injection of 2.4 mg/Kg buprenorphine solution in mice. As shown in FIG. 2, the analgesic effect lasted for 9 hours after the intravenous bolus injection.

TABLE 1

| | | Plasma Concentration of |
|---|---|---|
| Time (hours) | % MPE | buprenorphine (mg/ml) |
| 0.08 | 0 | 1138 |
| 0.25 | 53.7 | 407 |
| 0.5 | 96.3 | 207 |
| 1 | 100 | 91.2 |
| 2 | 77.4 | 31.5 |
| 3 | 93.3 | 22.7 |
| 5 | 100 | 13.0 |
| 7 | 72.7 | 7.1 |
| 9 | 54.4 | 5.2 |
| 12 | 8.1 | 1.9 |
| 18 | 0 | 0.58 |
| 24 | 0 | 0.21 |

### EXAMPLE 3

### Buprenorphine Formulations

Seven buprenorphine formulations (buprenorphine suspension in 0.75% Na CMC (0.75% w/v solution of Na CMC in R.O. water), PEG 400, TEC, ATEC, TBC, ATBC, and sesame oil) were investigated in 42 mice (6 mice per formulation). Each of the formulations contained 6.67% w/w buprenorphine and 93.33% w/w of the vehicle. Each mouse received about 60 μL of buprenorphine suspension, containing approximately 4 mg of buprenorphine base, by a subcutaneous injection. The analgesic effect in the animals was then was measured at the predetermined time intervals, namely 2 hours, 12 hours, day 1, day 1.5, day 2, day 2.5, day 3, day 3.5, day 4, day 4.5, day 5, day 5.5, and day 6. The animals were sacrificed on day 6 by cervical dislocation after anesthetized by isofluorine.

Blood was collected from the animals by cardiac puncture after sacrificing the mice and kept in heparinized centrifuge tube (1.5 mL). The blood was then centrifuged at 16000×g (14000 rpm) for 20 minutes and the plasma was transferred into 0.5 mL centrifuge tube, which was kept at −70° C. until analysis by LC/MS/MS method. The injection site was shaved and the skin was carefully dissected to reveal any residue left at the injection site. The drug residue left at the injection site in each mouse was quantified by an HPLC assay.

Table 2 shows the amount of buprenorphine that was administered into mice in each group, the amount of buprenorphine that remained at the injection site at the end of the study, the amount of buprenorphine that was released over the duration of study, percentage release of buprenorphine from the injection site during the study, and the buprenorphine plasma concentration at the end of the study.

TABLE 2

| | | | | | Buprenorphine |
|---|---|---|---|---|---|
| | Buprenorphine | Buprenorphine | | | plasma |
| | injected in | remaining | | | concentration |
| | each mouse | after 6 days | Buprenorphine | % | at the end of |
| | (mg) (Mean ± | (mg) (Mean ± | released (mg) | released | study (ng/ml) |
| FORMULATION | SD) | SD) | (Mean ± SD) | (Mean ± SD) | (Mean ± SD) |
| Na CMC | 5.35 ± 1.00 | 2.31 ± 1.16 | 3.05 ± 1.84 | 53.97 ± 26.62 | 39.3 ± 20.7 |
| PEG 400 | 5.61 ± 1.04 | 2.55 ± 0.55 | 3.06 ± 0.92 | 54.06 ± 9.50 | 70.1 ± 23.3 |
| TEC | 5.22 ± 0.94 | 1.88 ± 0.75 | 3.34 ± 1.46 | 62.02 ± 19.57 | 43.5 ± 17.6 |
| ATEC | 3.99 ± 0.38 | 1.83 ± 1.22 | 2.16 ± 1.02 | 55.21 ± 27.49 | 42.1 ± 11.1 |
| TBC | 4.56 ± 0.50 | 1.12 ± 0.32 | 3.44 ± 0.52 | 75.3 ± 7.4 | 37.1 ± 14.3 |
| ATBC | 5.61 ± 0.49 | 0.10 ± 0.05 | 5.51 ± 0.47 | 98.2 ± 0.70 | 30.7 ± 13.6 |
| Sesame | 5.82 ± 0.66 | 0.09 ± 0.08 | 5.73 ± 0.73 | 98.3 ± 1.6 | 27.1 ± 17.2 |
| Oil | | | | | |

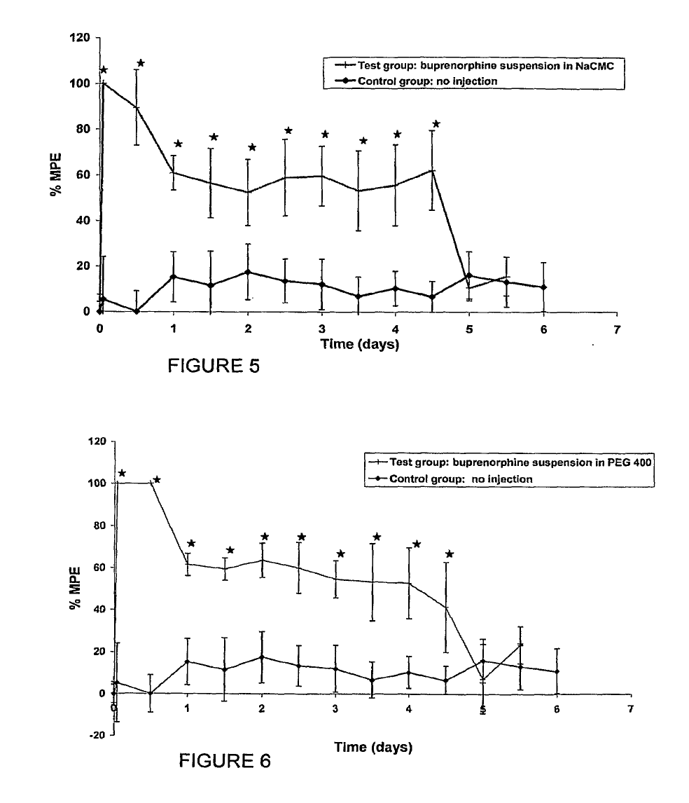

The analgesic effect achieved by the different formulations is shown in FIGS. 5 to 11. FIG. 5 shows that the buprenorphine suspension containing Na CMC achieved analgesic effect for 4.5 days by a single subcutaneous injection in mice. The % MPE ranged from 52˜100% for 4.5 days, after which it fell to 10˜16% MPE until the end of the study (6 days).

FIG. 6 shows that the buprenorphine suspension in PEG 400 achieved analgesic effect for 4.5 days in mice. The % MPE ranged from 41˜100% during initial 4.5 days, and then fell to 7˜23% MPE until the end of the study (6 days).

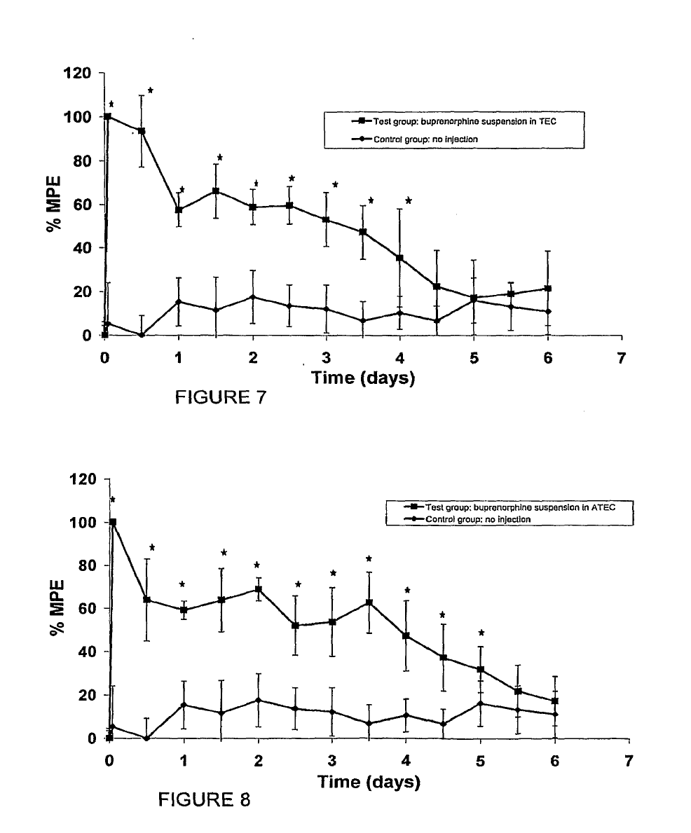

FIG. 7 shows that the buprenorphine suspension in TEC achieved analgesic effect for 4 days in mice. The % MPE ranged from 35˜100% during initial 4 days, and then fell to 17˜22% until the end of the study (6 days).

FIG. 8 shows that the buprenorphine suspension in ATEC achieved analgesic effect for 5 days in mice. The % MPE ranged from 32˜100% during initial 5 days, and then fell to 17˜22% until the end of the study (6 days).

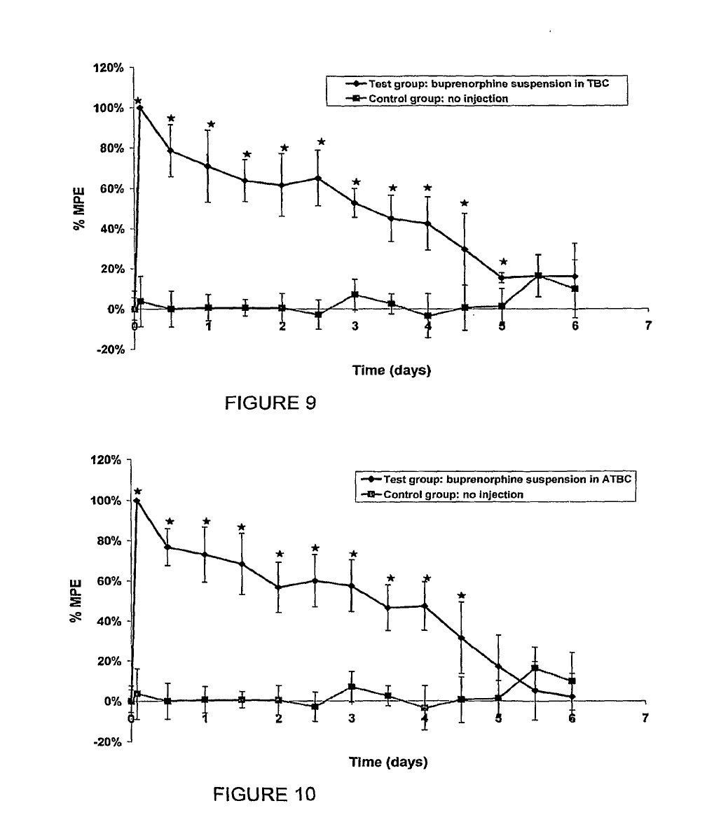

FIG. 9 shows that the buprenorphine suspension in TBC achieved analgesic effect for 5 days in mice. The % MPE ranged from 15˜100% during initial 5 days, and then fell to approximately 16% until the end of the study (6 days).

FIG. 10 shows that the buprenorphine suspension in ATBC achieved analgesic effect for 4.5 days in mice. The % MPE ranged from 31˜100% during initial 4.5 days, and then fell to 2˜17% until the end of the study (6 days).

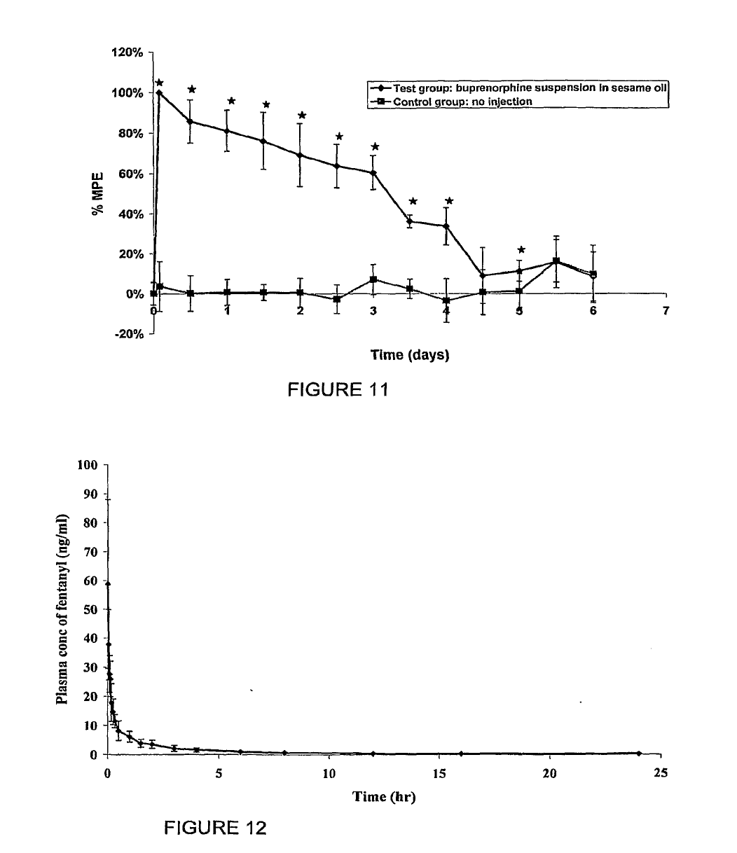

FIG. 11 shows that the buprenorphine suspension in sesame oil achieved analgesic effect for 4 days in mice. The % MPE ranged from 34˜100% during initial 4.5 days, and then fell to 9˜16% until the end of the study (6 days).

Overall, each of the formulations tested achieved an analgesic effect in mice for 4 to 5 days and met the target duration.

Drug residue remaining at the injection site in mice 6 days after subcutaneous injection of buprenorphine suspension in Na CMC, PEG 400, TEC, ATEC and TBC appeared as a solid. This means that the suspending vehicles were absorbed completely, and only buprenorphine was left as a solid residue. The drug residue left at the injection site in mice 6 days after a subcutaneous injection of buprenorphine suspension in ATBC and sesame oil appeared as liquid. This indicates that at least a portion of the suspending vehicles remained at the injection site 6 days after injection.

The result of quantitation of drug residue collected from the injection site is shown in Table 2. The in vivo release of buprenorphine (quantitated by measuring the amount of drug remaining in the residue at the injection site) from different formulations was different. For example, almost 98% of the drug was absorbed from the buprenorphine suspension in ATBC and sesame oil, whereas only 54% of the drug was absorbed from buprenorphine suspensions in Na CMC and PEG 400.

### EXAMPLE 4

### Preparation of Concentrated Fentanyl Base Solution in Tetraglycol (Glycofurol)

One gram of fentanyl base (Tyco Healthcare, Batch No: H01975) was accurately weighed and transferred to an appropriate container. 2.67 ml of tetraglycol (glycofurol) was added to the fentanyl powder and the resulting mixture was sonicated for 10 min until all the fentanyl powder was dissolved.

### EXAMPLE 5

### Preparation of Concentrated Fentanyl Base Solution in a Mixture of 95% Tetraglycol and 5% Water

One gram of fentanyl base was accurately weighed and transferred to an appropriate container. 3.20 ml of a mixture of 95% (v/v) Tetraglycol and 5% (v/v) of reverse osmosis (R/O) water was added to the fentanyl powder and the resulting mixture was sonicated for 10 min until all the fentanyl powder was dissolved.

### EXAMPLE 6

### Preparation of Concentrated Fentanyl Base Solution in a Mixture of 70% Tetraglycol and 30% Water

One gram of fentanyl base was accurately weighed and transferred to an appropriate container. Twenty five milliliters of a mixture of 70% (v/v) Tetraglycol and 30% (v/v) of reverse osmosis (R/O) water were added to the fentanyl powder and the resulting mixture was sonicated for 10 min until all the fentanyl powder was dissolved.

### EXAMPLE 7

### Preparation of Concentrated Fentanyl Base Solution in the Mixture of 90% PEG 400 and 10% Water

One gram of fentanyl base was accurately weighed and transferred to an appropriate container. Twenty five milliliters of a mixture of 90% (v/v) polyethylene glycol (PEG) 400 and 10% (v/v) of reverse osmosis (R/O) water were added to the fentanyl powder and the resulting mixture was sonicated for 10 min until all fentanyl powder was dissolved.

### EXAMPLE 8

### Preparation of Fentanyl Base Solution in Acetyl Tributyl Citrate (ATBC)

One gram of fentanyl base was accurately weighed and transferred to an appropriate container. Twenty five milliliters of acetyl tributyl citrate (ATBC) were added to the fentanyl powder and the resulting mixture was sonicated for 10 min until all the fentanyl powder was dissolved.

### EXAMPLE 9

### Preparation of Concentrated Fentanyl Base Solution in Acetyl Triethyl Citrate (ATEC)

One gram of fentanyl base was accurately weighed and transferred to an appropriate container. 6.67 ml of acetyl triethyl citrate (ATEC) was added to the fentanyl powder and the resulting mixture was sonicated for 10 min until all the fentanyl powder was dissolved.

### EXAMPLE 10

### Preparation of Concentrated Fentanyl Base Solution in Triethyl Citrate (TEC)

One gram of fentanyl base was accurately weighed and transferred to an appropriate container. Four milliliters of triethyl citrate (TEC) were added to the fentanyl powder and the resulting mixture was sonicated for 10 min until all the fentanyl powder was dissolved.

### EXAMPLE 11

### Intravenous Study (Prior Art)

Fentanyl citrate solution was administered intravenously (i.v.) to dogs (with an average weight of 19.5 Kg) at a dose of 50 microgram/kg. Blood samples were withdrawn from the jugular vein via a jugular catheter at the predetermined time intervals after i.v. injection. The drug content in the plasma was analyzed using a validated LC/MS method. FIG. 12 shows the plasma concentration of fentanyl after an intravenous injection of 50 microgram/kg of fentanyl in the dogs. Each data point on the graph represents the mean and standard deviation of fentanyl plasma concentrations from 6 dogs.

### EXAMPLE 12

### Administration of Concentrated Fentanyl Base Solution Prepared in Example 6 to dogs (Dose: 4 mg of Fentanyl Per Dog)

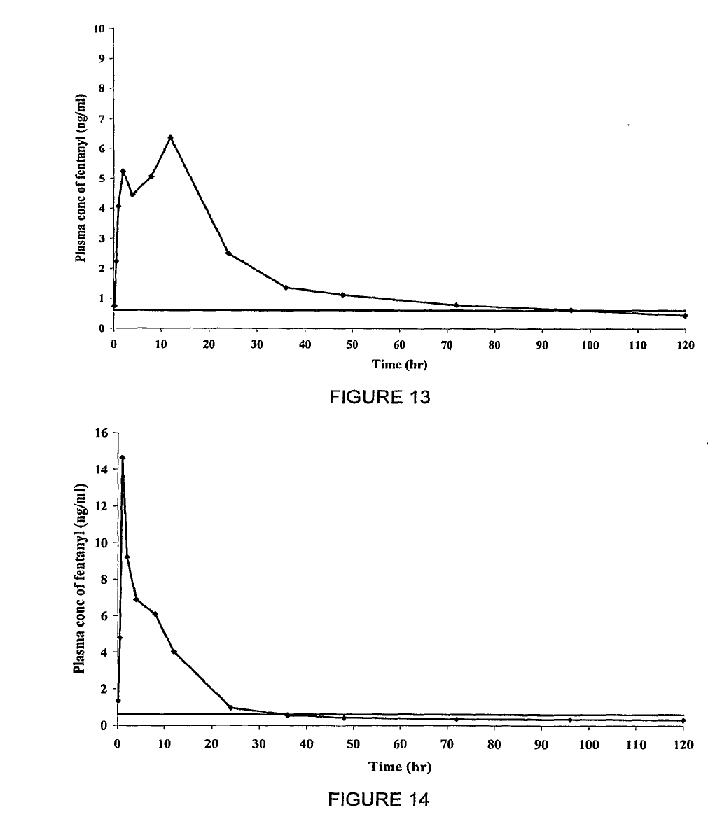

Concentrated fentanyl base solution in a mixture of 70% Tetraglycol and 30% water (fentanyl base solution prepared in Example 6) was administered subcutaneously (s.c.) to each dog at a dose of 4 mg per dog. Blood samples were withdrawn from the jugular vein via a jugular catheter at predetermined time intervals after the s.c. injection. The drug content in the plasma was analyzed using a validated LC/MS/MS method. FIG. 13 shows the plasma concentration of fentanyl after a subcutaneous injection of 4 mg of fentanyl in the dogs. Each data point on the graph represents the mean of fentanyl plasma concentrations from 2 dogs. As shown in FIG. 13, fentanyl plasma concentrations were maintained above 0.6 ng/ml in the dogs for at least 72 hours.

### EXAMPLE 13

### Administration of Concentrated Fentanyl Base Solution Prepared in Example 7 to Dogs (Dose: 4 mg of Fentanyl Per Dog)

Concentrated fentanyl base solution in a mixture of 90% PEG 400 and 10% water (fentanyl base solution prepared in Example 7) was administered subcutaneously to each dog at a dose of 4 mg per dog. Blood samples were withdrawn from the jugular vein via a jugular catheter at predetermined time intervals after the s.c. injection. The drug content in the plasma was analyzed using a validated LC/MS/MS method. FIG. 14 shows the plasma concentration of fentanyl after a subcutaneous injection of 4 mg of fentanyl in the dogs. Each data point on the graph represents the mean of fentanyl plasma concentrations from 2 dogs. As shown in FIG. 14, fentanyl plasma concentrations were maintained above 0.6 ng/ml in the dogs for at least 24 hours.

### EXAMPLE 14

### Administration of Fentanyl Base Solution Prepared in Example 8 to Dogs (Dose: 4 mg of Fentanyl Per Dog)

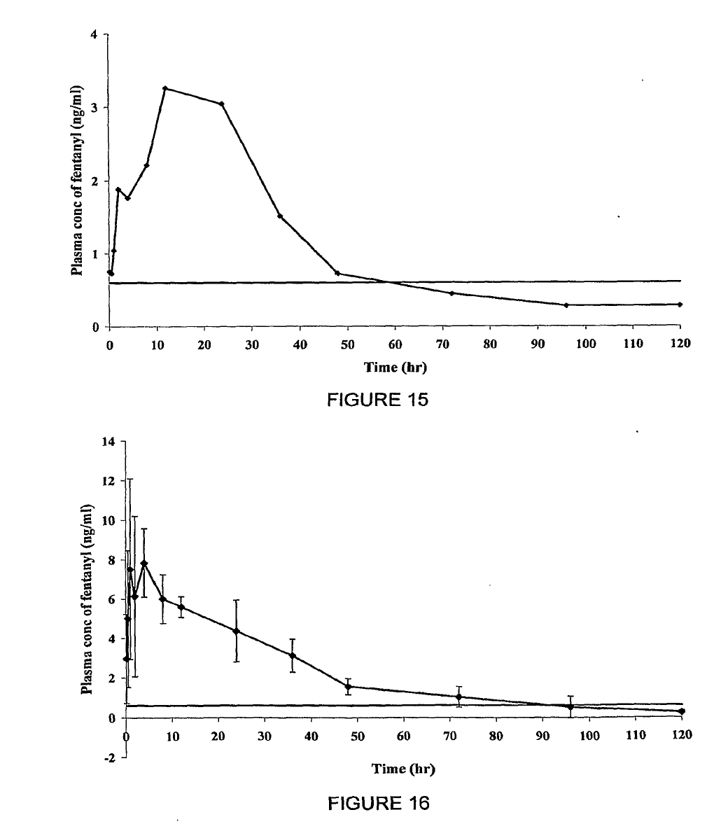

Fentanyl base solution in ATBC (fentanyl base solution prepared in Example 8) was administered subcutaneously to each dog at a dose of 4 mg per dog. Blood samples were withdrawn from the jugular vein via a jugular catheter at predetermined time intervals after s.c. injection. The blood samples were centrifuged and the resulting plasma was decanted into plastic vials and frozen until analysis. The drug content in the plasma was analyzed using a validated LC/MS/MS method. FIG. 15 shows the plasma concentration of fentanyl after a subcutaneous injection of 4 mg of fentanyl in the dogs. Each data point on the graph represents the mean of fentanyl plasma concentrations from 2 dogs. As shown in FIG. 15, fentanyl plasma concentrations were maintained above 0.6 ng/mL in the dogs for at least 48 hours.

### EXAMPLE 15

### Administration of Concentrated Fentanyl Base solution prepared in Example 6 to Dogs (Dose: 8 mg of Fentanyl Per Dog)

Concentrated fentanyl base solution in the mixture of 70% Tetraglycol and 30% water (Fentanyl base solution prepared in Example 6) was administered subcutaneously to each dog at a dose of 8 mg per dog. Blood samples were withdrawn from the jugular vein via a jugular catheter at predetermined time intervals after s.c. injection. The drug content in the plasma was analyzed using a validated LC/MS/MS method. FIG. 16 shows the plasma concentration of fentanyl after a subcutaneous injection of 8 mg of fentanyl in the dogs. Each data point on the graph represents the mean and standard deviations of fentanyl plasma concentrations from 3 dogs. As shown in FIG. 16, fentanyl plasma concentrations were maintained above 0.6 ng/mL in the dogs for at least 72 hours.

### EXAMPLE 16

### Administration of Fentanyl Base Solution Prepared in Example 8 to Dogs (Dose: 8 mg of Fentanyl Per Dog)

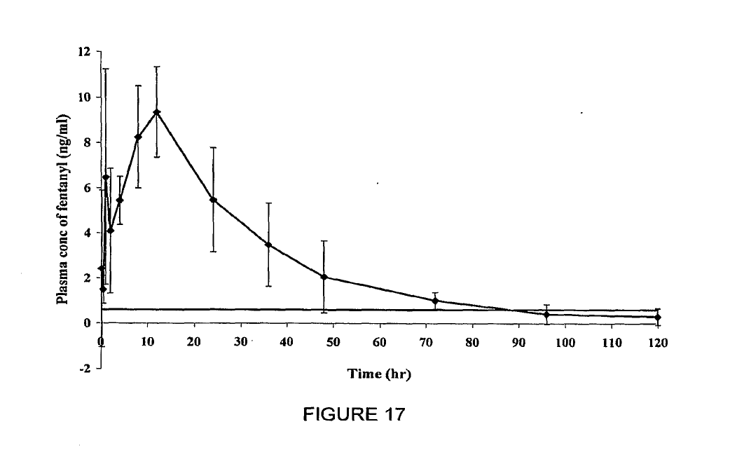

Fentanyl base solution in ATBC (Fentanyl base solution prepared in Example 8) was administered subcutaneously to each dog at a dose of 8 mg per dog. Blood samples were withdrawn from the jugular vein via a jugular catheter at predetermined time intervals after s.c. injection. The drug content in the plasma was analyzed using a validated LC/MS/MS method. FIG. 17 shows the plasma concentration of fentanyl after a subcutaneous injection of 8 mg of fentanyl in the dogs. Each data point on the graph represents the mean and standard deviations of fentanyl plasma concentrations from 3 dogs. As shown in FIG. 17, fentanyl plasma concentrations were maintained above 0.6 ng/mL in the dogs for at least 72 hours.

While preferred embodiments of the invention have been described in detail, it will be apparent to those skilled in the art that the disclosed embodiments may be modified. It is intended that such modifications be encompassed in the following claims. Therefore, the foregoing description is to be considered to be exemplary rather than limiting, and the scope of the invention is that defined by the following claims.

1. A method for inducing long-term analgesia in an animal, comprising parenterally and extravascularly administering into the body of an animal a composition comprising an analgesic medication of low water solubility that is dissolved, suspended, or emulsified in a solvent system, and permitting the medication to precipitate and/or partition in situ.

2. The method of claim 1 wherein the medication is in solution in a solvent that is miscible with aqueous bodily fluids.

3. The method of claim 1 wherein the medication is in suspension in a solvent other than water that is miscible with aqueous bodily fluids.

4. The method of claim 1 wherein the medication is in solution in a solvent that is immiscible with aqueous bodily fluids.

5. The method of claim 1 wherein the medication is in suspension in a solvent other than oil that is immiscible with aqueous bodily fluids.

6. The method of claim 1 wherein the analgesic medication is an opioid analgesic.

7. The method of claim 6 wherein the opioid analgesic is buprenorphine or fentanyl.

8. The method of claim 1 wherein the administration is by subcutaneous injection.

9. The method of claim 1 wherein the composition is a suspension and which method further comprises the steps of producing the suspension by obtaining a solution of the medication in a water-miscible solvent and combining into the solution a second solvent in which the medication is not soluble in a quantity sufficient to cause precipitation of the medication from the solution.

10. The method of claim 9 wherein the second solvent is water.

11. The method of claim 9 wherein the combining is by admixing the second solvent in vitro.

12. The method of claim 9 wherein the combining is by exposing the solution to aqueous body fluids following parenteral extravascular administration of the solution to an animal.

13. A sustained release composition for long-term analgesia in an animal comprising an analgesic medication of low water solubility and a solvent system in which the medication is dissolved, suspended, or emulsified, wherein the composition is situated in situ in a parenteral and extravascular location within the body of an animal.

14. The method of claim 13 wherein the medication is in solution in a solvent that is miscible with aqueous bodily fluids.

15. The method of claim 13 wherein the medication is in suspension in a solvent other than water that is miscible with aqueous bodily fluids.

16. The method of claim 13 wherein the medication is in solution in a solvent that is immiscible with aqueous bodily fluids.

17. The method of claim 13 wherein the medication is in suspension in a solvent other than oil that is immiscible with aqueous bodily fluids.

18. The composition of claim 13 wherein the analgesic medication is an opioid analgesic.

19. The composition of claim 18 wherein the opioid analgesic is buprenorphine or fentanyl.

20. The composition of claim 19 wherein the opioid analgesic is buprenorphine.

21. The composition of claim 20 wherein the solvent system comprises vegetable oil.

22. The composition of claim 21 wherein the solvent systems consists of vegetable oil.

23. The composition of claim 13 wherein the medication is in the form of a free acid, a free base, or a water-insoluble salt.

24. A method for obtaining a suspension of an analgesic medication of low water solubility comprising obtaining a solution of the medication in a water miscible solvent and exposing the solution to a second solvent in which the medication is not soluble in a quantity sufficient to cause precipitation of the medication from the solution, thereby obtaining a suspension.

25. The method of claim 24 wherein the exposing of the solution to the second solvent is in vitro.

26. The method of claim 24 wherein the exposing of the solution to the second solvent is by exposing the solution to aqueous body fluids following parenteral extravascular administration of the solution to an animal.

27. The method of claim 24 wherein the second solvent is water.

28. A method for obtaining a depot of an analgesic medication of low water solubility comprising obtaining a solution of the medication in a water miscible solvent and exposing the solution to a body fluid by administering parenterally and extravascularly the solution.

29. A method for obtaining a depot of an analgesic medication of low water solubility comprising obtaining a solution of the medication in a water immiscible solvent and administering the solution parenterally and extravascularly.
